(19) United States
(12) Patent Application Publication
(10) Pub. No.: US 2014/0358135 A1
(43) Pub. Date: Dec. 4, 2014

(54) METHOD AND APPARATUS FOR USING PHONOMYOGRAPHY TO PREVENT NERVE DAMAGE DURING A MEDICAL PROCEDURE

(71) Applicant: MEDTRONIC CRYOCATH LP, Toronto (JP)

(72) Inventors: Aleksandre T. SAMBELASHVILI, Maple Grove, MN (US); Nicolas COULOMBE, Anjou (CA)

(73) Assignee: MEDTRONIC CRYOCATH LP, Toronto (JP)

(21) Appl. No.: 13/904,138

(22) Filed: May 29, 2013

(51) Int. Cl. A61B 18/12; A61B 18/02

(52) U.S. Cl. CPC A61B 18/1233; A61B 18/02
USPC 606/21; 606/34

(57) ABSTRACT

A method and system for preventing phrenic nerve injury during a cardiac ablation procedure. The method generally includes stimulating a phrenic nerve with a pacing electrode at a location proximate a target treatment location, ablating tissue at the target treatment location with a treatment element, obtaining a heart audio signal with an audio sensor positioned proximate the target treatment location, and determining whether phrenic nerve stimulation (PNS) is present based as least in part on the obtained heart audio signal. A processor may be used to compare an amplitude of the obtained heart audio signal to a predetermined threshold amplitude. The processor may determine that PNS is present if the amplitude of the obtained audio signal is greater than the threshold amplitude. If PNS is present, ablation of the tissue may continue at the current or greater energy level and/or at the current treatment location without injuring the phrenic nerve.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The present invention relates to a method and system for performing a cardiac ablation procedure, such as a procedure used for treating atrial fibrillation, without injuring the phrenic nerves.

### BACKGROUND OF THE INVENTION

Cardiac arrhythmia, a condition in which the heart's normal rhythm is disrupted, includes many different forms. For example, cardiac arrhythmia includes premature atrial contractions (PACs), atrial flutter, accessory pathway tachycardias, atrial fibrillation, atrioventricular (AV) nodal reentrant tachycardia (AVNRT), premature ventricular contractions (PVCs), ventricular tachycardia (VT), ventricular fibrillation, long QT syndrome, and bradyarrhythmias.

Certain types of cardiac arrhythmias, including atrial fibrillation (AF), may be treated by ablation (for example, radiofrequency (RF) ablation, cryoablation, ultrasound ablation, laser ablation, and the like), either endocardially or epicardially. However, one of the possible complications of this procedure is damage to the phrenic nerves, which are involved in breathing and both receive and transmit nerve signals to the diaphragm. Phrenic nerve injury (PNI) can cause dyspnea, cough, hiccup, and/or sudden diaphragmatic elevation. The majority of patients with PNI recover over time, such as within days or months, but PNI may be persistent in a minority of patients.

The main function of the phrenic nerves is to control breathing by acting on the diaphragm. As such, it is possible to monitor phrenic nerve viability by stimulating the phrenic nerves electrically and detecting the corresponding physiologic response. Means for detecting physiologic response may include electromyography (the detection of electromyograms, or electrical potential generated by muscle cells when the cells are electrically or neurologically activated), mechanomyography (the detection of mechanomyograms, or mechanical signals observable from the surface of a muscle when the muscle is contracted), and kinemyography (the detection of electrical currents generated after deformation of a mechanosensor). However, these physiologic indices have drawbacks. Namely, electromyography is prone to noise and electromagnetic interference, whereas mechanomyography and kinemyography have limited sensitivity to sharp thoracic contractions.

It is therefore desired to provide a method and system for monitoring and/or preventing phrenic nerve injury during a medical procedure such as ablation.

### SUMMARY OF THE INVENTION

The present invention advantageously provides a method and system for preventing phrenic nerve injury during a cardiac ablation procedure. In a first embodiment, the method may generally include activating a pacing electrode proximate a phrenic nerve to transmit stimulation energy to the phrenic nerve, obtaining an audio signal from the patient's thoracic region while stimulation energy is transmitted to the phrenic nerve, determining whether phrenic nerve stimulation is present based on a comparison between an amplitude of the obtained audio signal and a predetermined threshold audio signal amplitude, and ablating tissue within the patient's heart proximate the phrenic nerve while phrenic nerve stimulation is present. Ablating tissue within the patient's heart may be by a medical device having a treatment element that delivers ablation energy to the tissue at a treatment location, and the method may further include adjusting at least one of the ablation energy delivered by the treatment element, an output of the pacing electrode, and a location of the treatment element relative to the phrenic nerve when phrenic nerve stimulation is determined to be absent. For example, the ablation energy delivered by the treatment element may be reduced and/or the output of the pacing electrode may be increased when phrenic stimulation is determined to be absent. Additionally or alternatively, the ablation energy delivered by the treatment element may be maintained or increased when phrenic nerve stimulation is determined to be present. Alternatively, ablating tissue within the patient's heart may be by a medical device having a treatment element that withdraws energy from tissue at the treatment location, and at least one of the ablation energy delivered by the treatment element, an output of the pacing electrode, and a location of the treatment element relative to the phrenic nerve may be adjusted when phrenic nerve stimulation is determined to be absent. For example, energy withdrawn from the tissue at the treatment location by the treatment element may be reduced when phrenic nerve stimulation is determined to be absent and the energy withdrawn from the tissue, and the energy withdrawn may be maintained or increased when phrenic nerve stimulation is determined to be present. Phrenic nerve injury may be determined to be present when phrenic nerve stimulation is determined to be absent. Stimulation energy may have a frequency, and the method may further include determining a patient-specific threshold audio signal amplitude, comparing the amplitude of the audio signal received from the audio sensor to the threshold audio signal amplitude at the frequency of the stimulation energy. For example, phrenic nerve stimulation may be determined to be present when the amplitude of the audio signal received from the audio sensor is greater than the amplitude of the threshold audio signal amplitude. The method may further include retrievably storing information about the presence of phrenic nerve injury as a result of ablation, the information including at least one of a location of the treatment element when phrenic nerve injury was determined to be present and an ablation energy level delivered when the phrenic nerve injury was determined to be present, and this information may be used to determine a location for re-ablation.

In another embodiment, the method may generally include activating a treatment element in contact with target tissue, stimulating a phrenic nerve with a pacing electrode, obtaining a heart audio signal with an audio sensor, transmitting the heart audio signal from the audio sensor to a processor, executing an algorithm within the processor to determine whether phrenic nerve stimulation is present based on the obtained heart audio signal, and maintaining activation of the treatment element to ablate the target tissue when the processor determines phrenic nerve stimulation is present. The heart audio signal may have an amplitude. The method may further include determining a patient-specific threshold audio signal amplitude, executing an algorithm within the processor to compare the amplitude of the obtained heart audio signal to the threshold amplitude, and executing an algorithm within the processor to determine that phrenic nerve stimulation is present when the amplitude of the obtained heart audio signal is greater than the threshold amplitude. The treatment element may ablate the target tissue by radiofrequency ablation, phased radiofrequency ablation, cryoablation, microwave ablation, ultrasound ablation, and/or laser ablation. Further, the processor may determine that phrenic nerve injury is present when phrenic nerve stimulation is absent.

The system may generally include a treatment device defining a distal portion and including a treatment element and a pacing electrode coupled to the treatment device distal portion; an audio sensor coupled to one of the treatment device or an auxiliary device, the auxiliary device being at least one of located within the patient's body and attached to an outer surface of the patient's body, the audio sensor obtaining an audio signal from the patient's heart; and a processor in communication with the audio sensor, the processor including an algorithm for determining phrenic nerve stimulation when the pacing electrode is activated. The processor may determine whether phrenic nerve stimulation is present based at least in part on an amplitude of a heart sound signal obtained by the audio sensor, and the treatment element may be activated when the processor determines that phrenic nerve stimulation is present, the presence of phrenic nerve stimulation indicating an absence of phrenic nerve injury.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 shows a first embodiment of a system including a medical device having a treatment element and an audio sensor;

FIG. 2 shows a second embodiment of a system including a medical device having treatment elements and an audio sensor;

FIG. 3 shows a distal portion of an electrophysiology device;

FIG. 4A shows an implantable defibrillator housing, the defibrillator including an audio sensor;

FIG. 4B shows an example of thoracic audio signals obtained from an audio sensor within an implantable defibrillator;

FIG. 5A shows audio sensors externally attached to a patient's chest;

FIG. 5B shows examples of thoracic audio signals obtained from audio sensors attached to a patient's chest;

FIG. 6 shows a flow chart of a method for using phonomyography to prevent phrenic nerve damage during an ablation procedure; and

FIG. 7 shows audio signals for different stimulation outputs, the stimulation applied using a lead in the left ventricle of a patient's heart.

### DETAILED DESCRIPTION OF THE INVENTION

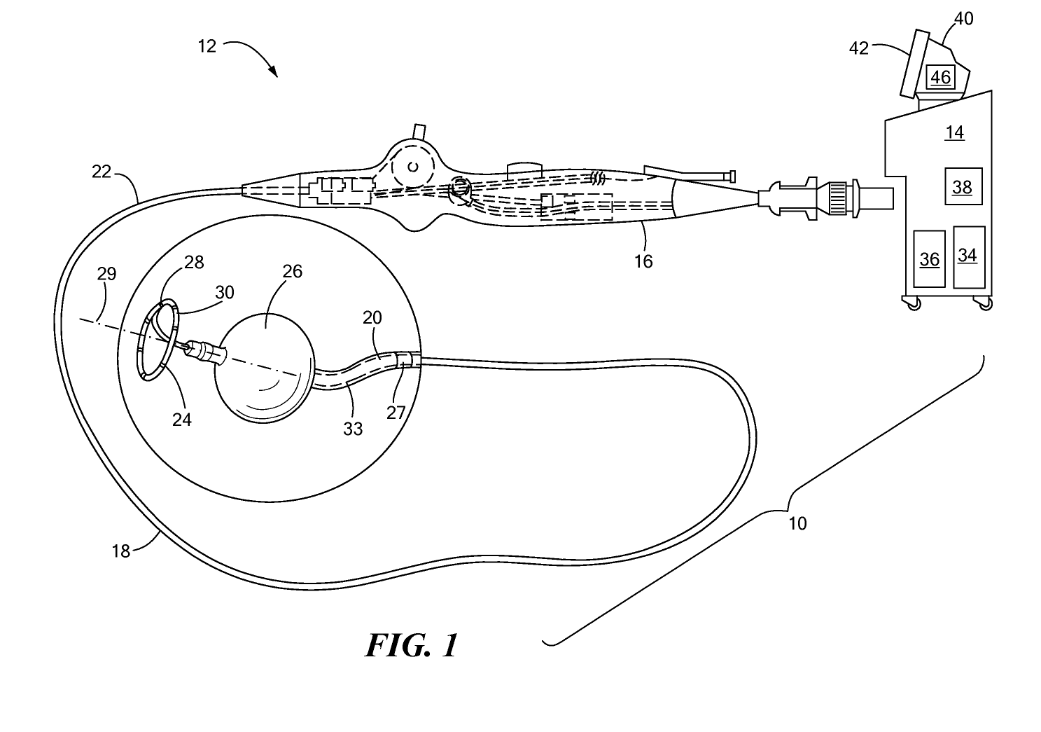

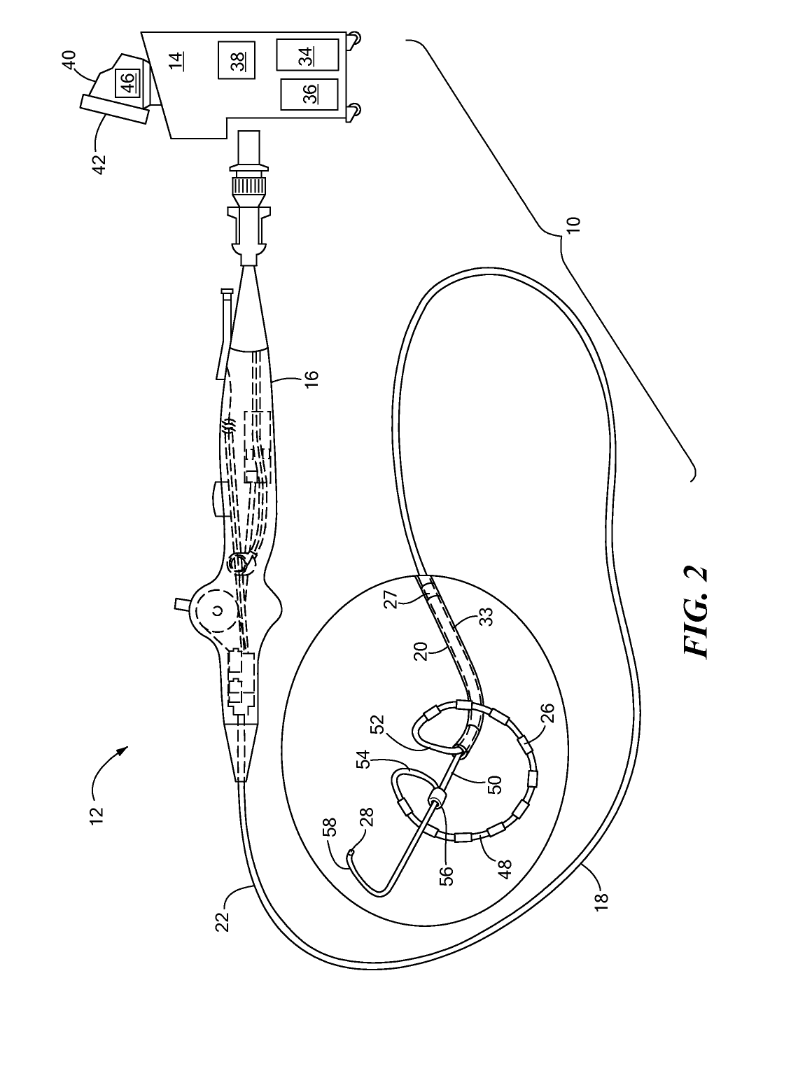

Referring now to FIG. 1, a system including a medical device having a treatment element and an audio sensor is shown. The system 10 generally includes a treatment device 12 for ablating tissue and a console 14 that houses various system 10 controls. The system 10 may be adapted for radiofrequency (RF) ablation and/or phased radiofrequency (PRF) ablation (as shown in FIG. 2), cryoablation, ultrasound ablation, laser ablation, microwave ablation, or other ablation methods or combinations thereof.

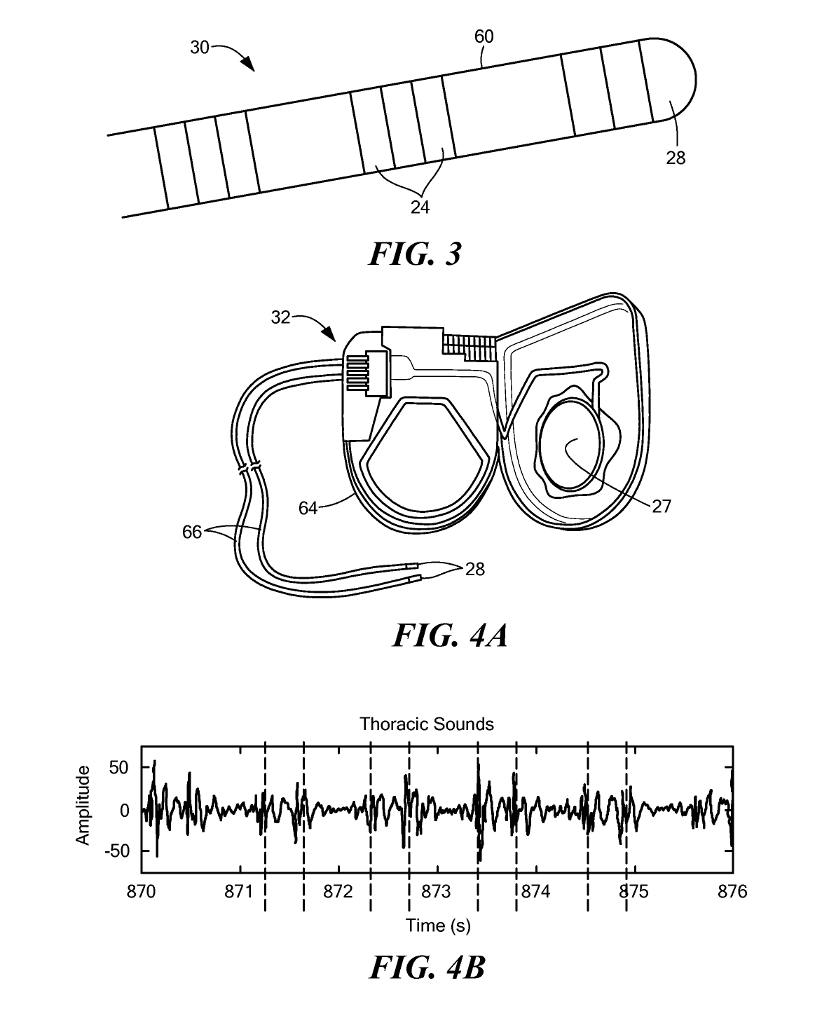

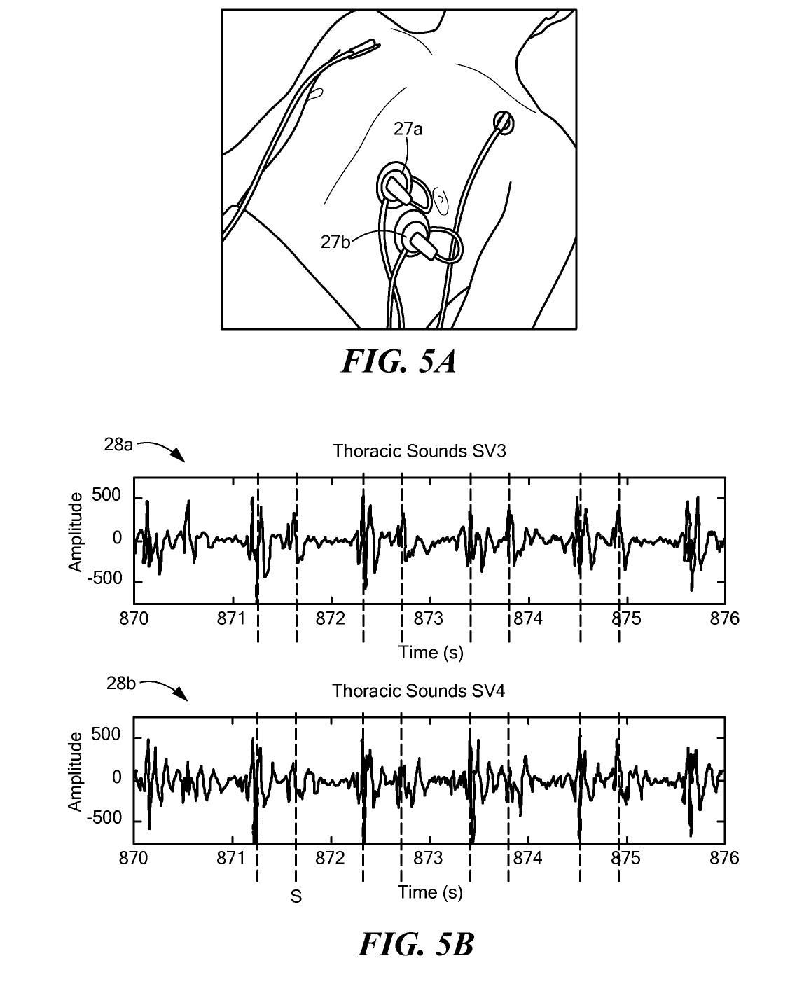

The treatment device 12 may be a catheter with ablation, mapping, and audio sensing capabilities. The treatment device 12 may additionally include pacing capabilities. As a non-limiting example, the treatment device 12 may generally include a handle 16, an elongate body 18 having a distal portion 20 and a proximal portion 22, one or more recording electrodes 24 for detecting electrophysiological signals, one or more treatment elements 26 for ablating or thermally treating tissue, and one or more audio sensors 27 for recording sounds generated from the thoracic region of the patient's body (for example, audio signals from the heart and/or diaphragm). Alternatively, the treatment device 12 may include one or more treatment elements 26 and optionally one or more audio sensors 27, whereas one or more recoding electrodes 24 and pacing electrodes 28 may be included on an electrophysiology catheter 30, which may be used as a component of the treatment device 12 (as shown in FIG. 1) or as an independent device (for example, as shown in FIG. 3) Likewise, the one or more audio sensors 27 may be included on a separate device (as shown in FIGS. 4A and 5A). The treatment device 12 may have a longitudinal axis 29. The one or more treatment elements 26 may be coupled to or disposed on at least a portion of the distal portion 20 of the elongate body 18. For example, the one or more treatment elements 26 may include an expandable element (such as a cryoballoon as shown in FIG. 1, an expandable array of electrodes as shown in FIG. 2, an expandable conductive mesh, and the like). Alternatively, the one or more treatment elements 26 may be borne directly on the distal portion 20 of the elongate body 18 (for example, the treatment device 12 may be a fixed-diameter catheter such as a focal catheter).

The one or more recording elements 24 may be sensors or electrodes capable of sensing and recording electrical activity within the myocardial cells as the cells polarize and depolarize. The one or more recording elements 24 may be coupled to or disposed on the distal portion 20 of the elongate body 18 of a medical device 12 with ablation capabilities (as shown in FIG. 1) Likewise, the one or more audio sensors 27 may be coupled to or disposed on the distal portion 20 of the elongate body 18 of a medical device with ablation capabilities (as shown in FIG. 1), or the one or more audio sensors 27 may be coupled to, disposed on, or disposed within a discrete secondary device, such as an electrophysiology catheter 30, an implantable device 32 such as a defibrillator (as shown in FIG. 4A), or on an external surface of a patient's chest (as shown in FIG. 5A).

The elongate body 18 of the treatment device 12 may include one or more lumens 33. If the treatment device 12 is a cryoablation catheter, for example, the elongate body 18 may include a main lumen, a fluid injection lumen in fluid communication with a coolant reservoir 34, and a fluid return lumen in fluid communication with a coolant return reservoir 36. In some embodiments, one or more other lumens may be disposed within the main lumen, may be disposed within the elongate body 18 along the longitudinal axis 29 parallel to the main lumen, and/or the main lumen may function as the fluid injection lumen or the fluid return lumen. If the treatment device 12 includes thermoelectric cooling elements or electrodes capable of transmitting RF (for example, the device shown in FIG. 2), ultrasound, microwave, electroporation energy, or the like, the elongate body 18 may include a lumen within which one or more wires are disposed, the wires being in electrical communication with one or more energy generators 38.

The console 14 may be in electrical and/or fluid communication with the treatment device 12 and may include one or more fluid (such as coolant or saline) reservoirs 34, fluid return reservoirs 36, energy generators 38 (for example, an RF or electroporation energy generator), and one or more computers 40 with displays 42, and may further include various other displays, screens, user input controls, keyboards, buttons, valves, conduits, connectors, power sources, and computers for adjusting and monitoring system 10 parameters. The computer 40 may be in electrical communication with the one or more treatment elements 26, the one or more recording electrodes 24, and/or the one or more audio sensors 27. Further, the computer 40 may include a processor 44 that includes one or more algorithms 46 executable to evaluate signals received from the one or more recording electrodes 24 and audio sensors 27 and to control, monitor, and/or suggest repositioning of the one or more treatment elements 26.

The treatment device 12 may be used in association with an electrophysiology catheter 30, with the treatment device 12 being used to ablate tissue and the electrophysiology catheter 30 being used to stimulate the phrenic nerve and, optionally, to record one or more electrophysiologic signals from the heart. The electrophysiology catheter 30 may, for example, be slidably disposed within a lumen 33 of the device 12 such that the electrophysiology catheter 30 may be positioned within the patient's anatomy independently before advancing the treatment device 12 over the electrophysiology catheter 30 to a treatment location (as shown in FIG. 1). Alternatively, the electrophysiology catheter 30 may be usable independently of the device 12 (as shown in FIG. 3). The electrophysiology catheter 30 may be flexible (for example, capable of being deflectable into a hooped shape (as shown in FIG. 1) and/or into a shape that includes one or more curves or bends. Further, the electrophysiology catheter 30 may include one or more pacing electrodes 28 that may be used to stimulate the phrenic nerve by transmitting energy in frequencies around approximately 100 Hz or more, for example, in a series of pacing pulses.

Referring now to FIG. 2, a second embodiment of a system including a medical device having treatment elements and an audio sensor is shown. The system 10 of FIG. 2 may generally be as shown and described in FIG. 1; however, the treatment device 12 may include one or more radiofrequency (RF) electrodes as the treatment elements 26 instead of the cryoballoon shown in FIG. 1. The RF electrodes 26, which may be composed of a conductive or selectively conductive material, may be used to ablate and/or thermally treat tissue through the transmission of RF and/or phased RF energy. As shown in the non-limiting embodiment of FIG. 2, the treatment device 12 may include a distal portion 20 that includes an adjustable carrier arm 48 on which the RF electrodes 26 may be disposed. The treatment device 12 may further include a shaft 50 that is slidably disposed within a lumen of the elongate body 18, with a first portion 52 of the carrier arm 48 being coupled to a distal portion 20 of the elongate body 18 and a second portion 54 of the carrier arm 48 being coupled to a distal portion 56 of the shaft 50. With this configuration, advancement or retraction of the shaft 50 within the elongate body 18 may adjust the diameter of the carrier arm 48. For example, the shaft 50 may be retracted to cause the carrier arm 48 to form a hooped portion suitable for ablating an area of tissue, for example, circumscribing a pulmonary vein ostium (such as a pulmonary vein antrum). Further, the treatment device 12 may include one or more audio sensors 27 for recording thoracic audio signals. The audio sensors 27 may be positioned at any location on the distal portion 20 of the treatment device 12; however, the audio sensors 27 should be located such that no other component of the treatment device 12 interferes with the reception of audio signals. The treatment device 12 may also include a guidewire 58 that is slidably disposed within a lumen of the shaft 50. The treatment device 12 may be used in association with an electrophysiology catheter 30 as a separate device (as shown in FIG. 3), with the treatment device 12 being used to ablate tissue and the electrophysiology catheter 30 being used to stimulate the phrenic nerve and, optionally, to record one or more electrophysiologic signals from the heart. Alternatively, the device 12 may further include one or more pacing electrodes 28. As a non-limiting example shown in FIG. 2, a pacing electrode 28 may be positioned on the guidewire 58, such as at the distal tip of the guidewire 58.

Referring now to FIG. 3, a distal portion of an electrophysiology device is shown. The electrophysiology (EP) device 30 may include one or more pacing electrodes 28 coupled to or disposed on a distal portion 60 of an EP catheter 30. Additionally, the EP catheter 30 may include one or more recording electrodes 24 for sensing electrical activity within the myocardial cells as the cells polarize and depolarize. Signals obtained by the one or more recording electrodes 24 may be used for mapping and/or determining the location of the device 30 within the heart. The EP catheter 30 may be positioned in proximity to and used to stimulate the phrenic nerve before, during, and/or after an ablation procedure using an ablation device suitable for use with, for example, radiofrequency (RF) ablation and/or phased radiofrequency (PRF) ablation (as shown in FIG. 2), cryoablation (as shown in FIG. 1), ultrasound ablation, laser ablation, microwave ablation, or other ablation methods or combinations thereof. Further, the electrophysiology catheter 30 shown in FIG. 3 may be used as a component of a treatment device 12 (as shown in FIG. 1). An audio sensor 27 may be, for example, a separate device (as shown in FIGS. 4A and 5A) or may be integrated with an ablation device (as shown in FIGS. 1 and 2).

Referring now to FIGS. 4A and 4B, an implantable defibrillator including an audio sensor and thoracic audio signals obtainable therefrom are shown. An implantable defibrillator (or “implantable cardioverter-defibrillator,” ICD) 32 is a small, battery-powered device that may be implanted within a patient and programmed to detect cardiac arrhythmia and respond by delivering electricity to the heart. The ICD 32 may be able to distinguish between ventricular fibrillation, ventricular tachycardia, atrial fibrillation, and other cardiac arrhythmias. For example, the ICD 32 may use heart beat rhythm, rate, and morphology discrimination to determine whether an irregular rhythm originates in the atria or the ventricles. As shown in FIG. 4A, the ICD 32 may include an audio sensor 27 within the housing 64 of the device for the detection of thoracic sounds (for example, the ICD 32 shown in FIG. 4A is open to expose the inner components, including the audio sensor 27). The audio sensor 27 may transmit recorded audio signals to the processor 44 for use such as described in FIG. 1. A non-limiting example of audio data recordable by the audio sensor 27 is shown in FIG. 4B. The audio sensor 27 and/or a computer or processor integrated within the ICD 32 may be capable of wirelessly transmitting data to the processor 44 so that the ICD 32 does not have to be accessed, plugged into, or otherwise manipulated or connected to, the processor 44. Additionally, the ICD 32 may include one or more leads 66 that include one or more pacing electrodes 28. These pacing electrodes 28 may be used to stimulate the phrenic nerve before, during, and/or after an ablation procedure using the treatment device 12. Thus, a preexisting ICD 32 may be used for the method of preventing phrenic nerve damage shown and described herein.

Referring now to FIGS. 5A and 5B, audio sensors externally attached to a patient's chest and thoracic audio signals obtainable therefrom are shown. Like the ICD 32 shown and described in FIGS. 4A and 4B, one or more audio sensors 27 may be removably attached to the outside of a patient's body in the thoracic region, such as on the chest or torso proximate the heart (as shown in FIG. 5A). As a non-limiting example, FIGS. 5A and 5B include the use of two audio sensors 27a, 27b. The one or more audio sensors 27 may transmit data to the processor 44 in either a wireless or wired configuration. A non-limiting example of audio data recordable by two audio sensors 27a, 27b is shown in FIG. 5B.

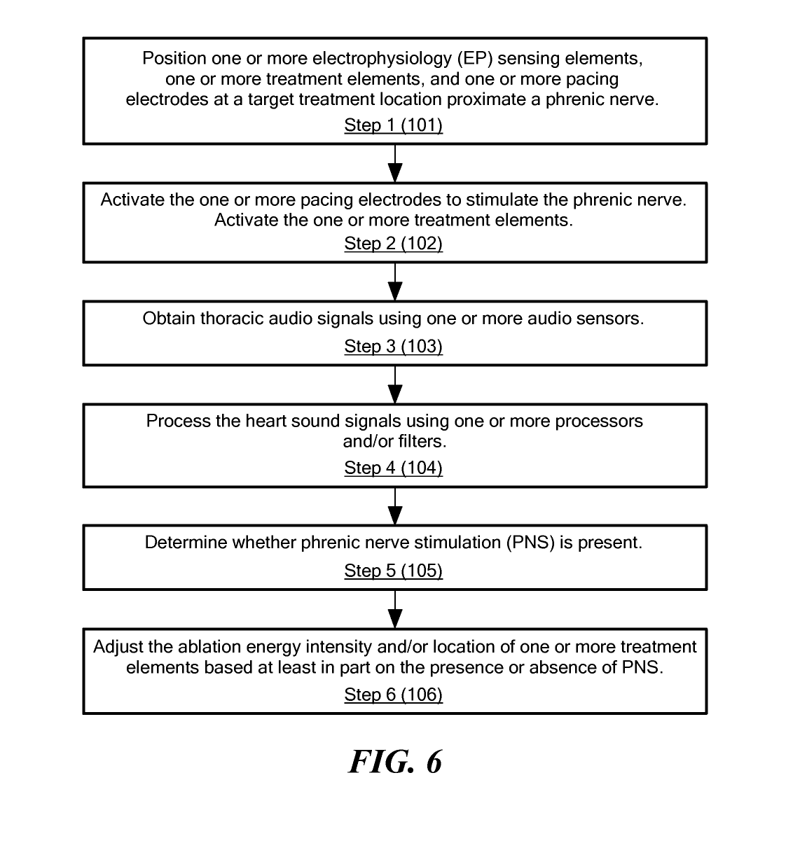

Referring now to FIG. 6, a flow chart of a method for using phonomyography to prevent phrenic nerve damage during an ablation procedure is shown. The method is based on the premise that phonomyography (a technique that measures the force of muscle contraction by recording the low frequency sounds created during muscular activity) may be used to detect phrenic nerve stimulation (PNS) during an ablation procedure, such as ablation used to treat atrial fibrillation. It the first step 101 of the method, one or more electrophysiologic sensing elements may be positioned within a patient's heart at a location proximate a phrenic nerve. For example, an EP catheter 30 having one or more recording electrodes 24, such as that shown in FIG. 3, may be used. Additionally, one or more pacing electrodes 28 may be positioned within the patient's heart at a location proximate a phrenic nerve. For example, the one or more pacing electrodes 28 may be positioned in the thoracic region, such as in the heart or in the esophagus. In general, the right phrenic nerve courses by the superior vena cava and close to the right superior pulmonary vein as well as the right inferior pulmonary veins on occasion, whereas the left phrenic nerve passes over the pericardium of the left ventricle. Both right and left phrenic nerves are in communication with the diaphragm. In the second step 102 of the method, the device with electrophysiologic sensing capabilities may be activated to stimulate a phrenic nerve. Which nerve is stimulated may depend on the target treatment location within the heart. Although FIG. 6 shows activation of the one or more treatment elements 26 of treatment device 12 in the second step 102, the second step 102 may be performed before, during, and/or after an ablation procedure.

In the third step 103 of the method, the one or more audio sensors 27 may be used to detect thoracic audio signals and transmit the signals to the processor 44. The one or more audio sensors 27 may be positioned at a location from which thoracic sounds may be detected. If, for example, the one or more audio sensors 27 are located on a device separate from the EP catheter 30, the amplitude of the sound signal in response to the stimulation may be used to optimize the location of the audio sensors 27. That is, the one or more audio sensors 27 may be repositioned if reception of thoracic audio signals is poor. In the fourth step 104 of the method, the obtained thoracic audio signals may be processed using the processor 44 and/or one or more filters. For example, the signals may be band-pass filtered or ensemble-averaged, and digital envelopes may be obtained and a threshold for the digital envelopes determined.

In the fifth step 105 of the method, the one or more processors 44 may determine whether phrenic nerve stimulation (PNS) is present. In general, the absence of PNS may be indicative of phrenic nerve injury (PNI). Under normal conditions, the frequency of slow diaphragmatic contractions is approximately 10-15 contractions per minute. If PNS is present, then the audio sensor signal will have a harmonic at the same frequency as the frequency of the pacing pulses. The one or more processors 44 may include a PNS detection algorithm that monitors the Fourier amplitude of the audio sensor signal at a frequency equal to the frequency of the pacing pulses, when the pulses are delivered. If the amplitude exceeds a predetermined threshold, which threshold may be patient specific, then the algorithm will determine that PNS is present. In this case, the pacing pulses may be temporarily stopped for a number of heart beats and the amplitude may be examined again. If, as a result, amplitude is detected that is below the predetermined threshold, the one or more processors 44 may determine that PNS is absent. PNS tests may be performed once within a certain period of time. In general, the detection of PNS may take place during delivery of energy to target tissue, but the automatic detection algorithm may be “trained” during periods when ablation energy is not being transmitted. That is, the phrenic nerve may be stimulated and the thoracic sounds recorded, then stimulation may be stopped and thoracic sounds recorded. This method may be used to obtain a threshold amplitude that is personal to the patient. A previously detected presence of PNS may affect the frequency of subsequent PNS tests. Information obtained from PNS testing (for example, presence or absence of PNS, energy delivery levels, activated electrodes, etc.) may be transmitted to a mapping system for visualization and/or recording. This information may be stored (for example, in a computer) for later access by the user, such as with the use of a mapping system. For example, the mapping system may be integrated within the system 10 or may be part of another system, and may include one or more processors including mapping software and algorithms, displays, user input devices, and the like. Further, the mapping system may be in communication with the one or more recording electrodes 24. The mapping system may use the information to identify “hot spots” or areas at which phrenic nerve injury occurred and to supply the user with information about, for example, the energy levels at which injury occurred. Thus, a user may avoid these locations and/or energy levels to prevent future phrenic nerve injury in the same patient.

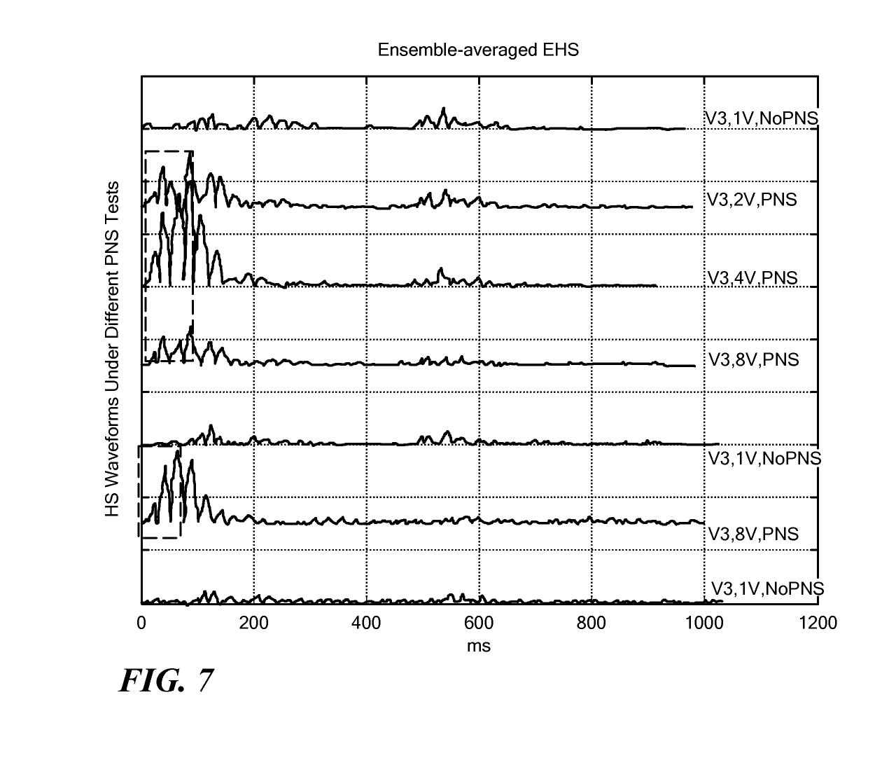

In the sixth step 106 of the method, if one or more consecutive PNS tests yield negative results (that is, if PNS is absent), the ablation energy delivery may be reduced (in a cryoablation system, the temperature of the treatment element may be allowed to increase) until a PNS test yields positive results (that is, until PNS is present). Additionally or alternatively, since PNS may be absent due to inadequate capture of the pacing pulses, the pacing output may be increased (as shown in FIG. 7). Conversely, if PNS is present, the ablation energy delivery may be maintained or increased (in a cryoablation system, the temperature of the treatment element may be maintained or allowed to decrease) as long as PNS continues to be present. If PNS disappears, an alert may be generated by the system and the treatment element 26 may be repositioned. For example, an audio alert or visual alert may be generated that signals the surgeon to manually reposition and/or reduce energy delivery. Alternatively, the system may automatically adjust the treatment element 26, such as by activating different electrodes on a multi-polar catheter and/or reducing energy delivery.

Referring now to FIG. 7, audio signals for different stimulation outputs are shown, the stimulation applied using a lead in the left ventricle of a patient's heart. In the non-limiting example shown in FIG. 7, PNS by left-ventricular pacing may be manifested in the early vibrations immediately following the stimulus pulse (time 0) and preceding the S1 thoracic sound. At a low pacing output of 1V, the PNS is absent, whereas PNS is present at pacing outputs above 1V. PNS is shown in FIG. 7 within the dashed-line boxes.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A method of preventing phrenic nerve injury, the method comprising:
activating a pacing electrode proximate a phrenic nerve to transmit stimulation energy to the phrenic nerve;
obtaining an audio signal from the patient's thoracic region while stimulation energy is transmitted to the phrenic nerve;
determining whether phrenic nerve stimulation is present based on a comparison between an amplitude of the obtained audio signal and a predetermined threshold audio signal amplitude; and
ablating tissue within the patient's heart proximate the phrenic nerve while phrenic nerve stimulation is present.

2. The method of claim 1, wherein the ablating tissue within the patient's heart is by a medical device having a treatment element that delivers ablation energy to the tissue at a treatment location, the method further comprising:
adjusting at least one of the ablation energy delivered by the treatment element, an output of the pacing electrode, and a location of the treatment element relative to the phrenic nerve when phrenic nerve stimulation is determined to be absent.

3. The method of claim 2, wherein the ablation energy delivered by the treatment element is reduced when phrenic stimulation is determined to be absent.

4. The method of claim 2, wherein the output of the pacing electrode is increased when phrenic stimulation is determined to be absent.

5. The method of claim 2, wherein the treatment element delivers at least one of radiofrequency energy, phased radiofrequency energy, microwave energy, ultrasound energy, and laser energy.

6. The method of claim 1, wherein the ablating tissue within the patient's heart is by a medical device having a treatment element that delivers ablation energy to tissue at the treatment location, the method further comprising:
maintaining or increasing the ablation energy delivered by the treatment element when phrenic nerve stimulation is determined to be present.

7. The method of claim 1, further comprising determining whether phrenic nerve injury is present, wherein phrenic nerve injury is determined to be present when phrenic nerve stimulation is determined to be absent.

8. The method of claim 1, wherein the ablating tissue within the patient's heart is by a medical device having a treatment element that withdraws energy from tissue at the treatment location, the method further comprising:
adjusting at least one of the ablation energy delivered by the treatment element, an output of the pacing electrode, and a location of the treatment element relative to the phrenic nerve when phrenic nerve stimulation is determined to be absent.

9. The method of claim 8, wherein the energy withdrawn from the tissue at the treatment location by the treatment element is reduced when phrenic nerve stimulation is determined to be absent and the energy withdrawn from the tissue is maintained or increased when phrenic nerve stimulation is determined to be present.

10. The method of claim 9, wherein the treatment element is a cryoballoon in fluid communication with a source of coolant.

11. The method of claim 1, wherein stimulation energy has a frequency, the method further comprising:
determining a threshold audio signal amplitude, the threshold audio signal amplitude being specific to the patient; and
comparing the amplitude of the audio signal received from the audio sensor to the threshold audio signal amplitude at the frequency of the stimulation energy.

12. The method of claim 11, wherein phrenic nerve stimulation is determined to be present when the amplitude of the audio signal received from the audio sensor is greater than the amplitude of the threshold audio signal amplitude.

13. The method of claim 7, further comprising:
retrievably storing information about the presence of phrenic nerve injury as a result of ablation, the information including at least one of a location of the treatment element when phrenic nerve injury was determined to be present and an ablation energy level delivered when the phrenic nerve injury was determined to be present.

14. The method of claim 13, the method further comprising:
re-ablating tissue with the treatment element within the patient's heart at a location determined at least in part on the information about the presence of phrenic nerve injury as a result of ablation.

15. The method of claim 1, wherein the audio sensor is at least one of coupled to the distal portion of the treatment device, located within an implantable device within the patient's body, coupled to the electrophysiology device, and in contact with an outer surface of the patient's body.

16. A method for ablating tissue, the method comprising:
activating a treatment element in contact with target tissue;
stimulating a phrenic nerve with a pacing electrode;
obtaining a thoracic audio signal with an audio sensor;
transmitting the thoracic audio signal from the audio sensor to a processor;
executing an algorithm within the processor to determine whether phrenic nerve stimulation is present based on the obtained thoracic audio signal; and
maintaining activation of the treatment element to ablate the target tissue when the processor determines phrenic nerve stimulation is present.

17. The method of claim 16, wherein the thoracic audio signal has an amplitude, the method further comprising:
determining a patient-specific threshold audio signal amplitude;
executing an algorithm within the processor to compare the amplitude of the obtained thoracic audio signal to the threshold amplitude; and
executing an algorithm within the processor to determine that phrenic nerve stimulation is present when the amplitude of the obtained thoracic audio signal is greater than the threshold amplitude.

18. The method of claim 17, wherein the treatment element ablates the target tissue by at least one of radiofrequency ablation, phased radiofrequency ablation, cryoablation, microwave ablation, ultrasound ablation, and laser ablation.

19. The method of claim 17, wherein the processor determines that phrenic nerve injury is present when phrenic nerve stimulation is absent.

20. A system for preventing phrenic nerve injury during ablation of a patient's heart, the system comprising:
a treatment device defining a distal portion and including a treatment element and a pacing electrode coupled to the treatment device distal portion;
an audio sensor coupled to one of the treatment device or an auxiliary device, the auxiliary device being at least one of located within the patient's body and attached to an outer surface of the patient's body, the audio sensor obtaining an audio signal from the patient's thoracic region; and
a processor in communication with the audio sensor, the processor including an algorithm for determining phrenic nerve stimulation when the pacing electrode is activated.

21. The system of claim 20, wherein the processor determines whether phrenic nerve stimulation is present based at least in part on an amplitude of a heart sound signal obtained by the audio sensor.

22. The system of claim 21, wherein the treatment element is activated when the processor determines that phrenic nerve stimulation is present, the presence of phrenic nerve stimulation indicating an absence of phrenic nerve injury.
